(19) United States
(12) Patent Application Publication
(10) Pub. No.: US 2008/0019933 A1
(43) Pub. Date: Jan. 24, 2008

(54) Method of making-up dark skin

(75) Inventors: Ludovic Thevenet, Bourg La Reine (FR); Gaetan Chevalier, Boigny Sur Bionne (FR); Frank Girier-Dufournier, Paris (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/878,070

(22) Filed: Jul. 20, 2007

Related U.S. Application Data
(60) Provisional application No. 60/836,690, filed on Aug. 10, 2006.
(60) Provisional application No. 60/836,692, filed on Aug. 10, 2006.
(60) Provisional application No. 60/837,938, filed on Aug. 16, 2006.
(60) Provisional application No. 60/837,920, filed on Aug. 16, 2006.
(60) Provisional application No. 60/837,939, filed on Aug. 16, 2006.
(60) Provisional application No. 60/837,940, filed on Aug. 16, 2006.
(60) Provisional application No. 60/838,143, filed on Aug. 17, 2006.
(60) Provisional application No. 60/838,141, filed on Aug. 17, 2006.
(60) Provisional application No. 60/838,140, filed on Aug. 17, 2006.

(30) Foreign Application Priority Data
Jul. 21, 2006 (FR) 06.06667
Jul. 21, 2006 (FR) 06.06669
Jul. 21, 2006 (FR) 06.06672
Jul. 21, 2006 (FR) 06.06665
Jul. 21, 2006 (FR) 06.06659
Jul. 21, 2006 (FR) 06.06661
Jul. 21, 2006 (FR) 06.06658
Jul. 21, 2006 (FR) 06.06664
Jul. 21, 2006 (FR) 06.06663

(51) Int. Cl. A61K 8/00; A61Q 19/00

(52) U.S. Cl. 424/63

(57) ABSTRACT

The present invention relates to a method of making-up dark skin, the method comprising the step consisting in applying to the skin a cosmetic composition comprising, in a cosmetically acceptable medium, at least one red interference pigment which, when the composition is applied to the skin, is capable of generating highlights with an intensity of 3000 cd·m⁻² or more and with a dominant wavelength in the range 580 nm to 650 nm.

This non provisional application claims the benefit of French Applications Nos. 06 06669, 06 06672, 06 06667, 06 06665, 06 06659, 06 06661, 06 06658, 06 06664, 06 06663 filed on Jul. 21, 2006 and U.S. of Provisional Applications Nos. 60/837,938, 60/837,920, 60/837,939, 60/837,940 filed on Aug. 16, 2006 and Nos. 60/836,690, 60/836,692 filed on Aug. 10, 2006 and Nos. 60/838,143, 60/838,141 and 60/838,140, filed on Aug. 17, 2006.

The present invention seeks to propose cosmetic compositions for application to the skin, and more particularly compositions for dark skin, including black and colored skin.

### SUMMARY

There exists a need to benefit from a makeup composition that is more particularly for dark skin, and that enables that type of skin to achieve a radiant color.

In one of its aspects, the present invention thus provides a method of making-up dark skin, the method comprising the step consisting in applying to the skin a cosmetic composition comprising, in a cosmetically acceptable medium, at least one red interference pigment which, when the composition is applied to the skin, is capable of generating highlights with an intensity of 3000 cd·m⁻² [candela per square meter] or more and with a dominant wavelength in the range 580 nm [nanometers] to 650 nm.

The invention makes it possible, on dark skin, to obtain a more beautiful makeup effect, in particular makeup that creates the perception of a warm tone by the particular color and light as a result of the shine of the pigment.

The composition may present saturation C* that is 30 or more, or even 35 or more.

The composition may present a hue angle h° in the range 10° to 30°, in particular in the range 15° to 25°.

The optical thickness (product of the thickness of the layer producing the interference by the index of refraction) of the red color interference pigment may range from 310 nm to 430 nm for interference of order 1 and from 620 nm to 860 nm for interference of order 2. These optical thicknesses cover the red color (from 620 nm 700 nm) for two orders of interference by taking into account a variation of the angle from 0 to 70° for a cosmetic medium having a refraction index ranging for example from 1.4 to 1.5.

The present invention also provides a makeup composition comprising, in a cosmetically acceptable medium, at least one colorant, and at least one red interference pigment which, when the composition is applied to the skin, is capable of generating highlights with an intensity of 3000 cd·m⁻² or more and with a dominant wavelength in the range 580 nm to 650 nm.

The colorant may have a dominant wavelength in the range 580 nm to 650 nm, so as to create an iridescent effect when the size of the red interference pigment is of the order of the resolving power of the eye.

In one of its aspects, independently of what precedes, the invention provides a cosmetic composition comprising, dispersed in a cosmetically acceptable medium, a red interference pigment that is capable of creating highlights with a dominant wavelength in the range 580 nm to 650 nm and with an intensity of 3500 cd·m⁻² or more when the composition is applied to a surface, the composition not containing, in the medium, white fillers or solid bodies that generate a color by absorption, or, when the composition does contains them, the total amount of such solid bodies being 1% or less by weight relative to the total weight of the composition.

This allows the color produced by the interference phenomenon to be clearly dominating compared to the color produced by adsorption and a bright red make-up may be obtained. In this aspect, the composition need not contain white fillers or diffusing pigments in the medium.

Moreover, the kind and the quantity of solid bodies other than the red interference pigment could be a function of the desired optical properties and textures, provided that the interference phenomenon responsible for the red highlights is not in any way deleteriously affected.

In another one of its aspects, independently of what precedes, the invention provides a cosmetic composition comprising, dispersed in a cosmetically acceptable medium:
at least one red interference pigment that is capable of creating highlights with a dominant wavelength in the range 580 nm to 650 nm and with an intensity of 3500 cd·m⁻² or more when the composition is applied to a surface,—reflective particles that are capable of generating, on said surface, other highlights with an intensity that is greater than or equal to the intensity of the red interference pigment, better greater than or equal to 4 000 cd m⁻².

This allows modifying the aspect of the composition without affecting the red color produced by the red interference pigment.

In particular, the above-mentioned reflective particles can be used in a relatively small amount while making it possible, by means of their reflectivity, to modify the clarity of the composition. In addition, reflective particles absorb less light than conventional diffusing pigments that generate a color by an absorption phenomenon.

In another one of its aspects, independently of what precedes, the invention provides a cosmetic composition comprising, in a cosmetically acceptable medium, a red interference pigment that, when the composition is applied to a support, is capable of generating highlights with an intensity of 3000 cd·m⁻² or more and with a dominant wavelength in the range 580 nm to 650 nm, the composition presenting a turbidity index of 100 nephelometric turbidity units (NTU) or less. This allows the color produced by the interference phenomenon to be clearly dominating compared to the color produced by adsorption for precise conditions of observation. When those conditions change, the color produced by adsorption can be observed by the observer.

In another one of its aspects, the invention provides a set of at least two cosmetic compositions comprising, dispersed in a cosmetically acceptable medium, at least one red interference pigment that, when the corresponding composition is applied to a surface, is capable of generating highlights with an intensity of 3000 cd·m⁻² or more and with a dominant wavelength in the range 580 nm to 650 nm, the saturation difference between two compositions of the set being 2 or less, the red interference pigment in said two compositions being at concentrations that differ by at least 1%.

The set may comprise more than two compositions and the above relationship may be satisfied, where appropriate, for any two compositions of the set or for only some of them.

Such a set of compositions makes it possible to have different concentrations of red highlights, and the Applicant has observed, in unexpected manner, that the presence of such an interference pigment having different concentrations does not lead to a significant modification in saturation.

The compositions can have substantially the same medium.

The term “substantially the same medium” means that the same compounds are found in the compositions, at concentrations that can vary as a function of the amount of red interference pigment.

Thus, the content of a compound may differ from one composition to another in order to compensate for the variation in the amount of red interference pigment.

The compositions need not include solid bodies other than the red interference pigment.

Between the two above-mentioned compositions of the set, the amount of red interference pigment can differ by at least 2%.

In what follows, the expression “the composition” may refer to any one of the compositions of the set.

In another of its aspects, the invention provides a cosmetic composition comprising, in a cosmetically acceptable medium:
an interference pigment that is red and that is capable of generating highlights with an intensity that is greater than or equal to 3000 cd·m⁻² and with a dominant wavelength in the range 580 nm to 650 nm; and
magnetic bodies presenting non-zero magnetic susceptibility.

The invention exploits the very particular sensitivity of the red interference pigment to its environment. Thus, by means of the presence of the interference pigment, even a small modification to the orientation and/or to the positioning of the magnetic bodies in the composition is likely, in the invention, to lead to an observable visual effect, e.g. a variation in the intensity and/or in the concentration of the highlights, in particular by means of the red interference pigment being masked to a greater or lesser extent by the magnetic bodies.

The composition can take on a state that prevents any new change in the orientation of the magnetic bodies under the effect of a magnetic field after a given drying time. This applies to a nail varnish, for example.

Alternatively, in some cases, the orientation of the magnetic bodies can be modified at any time, in particular when the composition does not dry or presents a drying time that is very long. This may apply to a foundation, for example.

By way of example, when the composition includes a volatile solvent, the magnetic field is exerted a short time after depositing the composition, so as to change its appearance before it dries.

Where appropriate, the magnetic bodies can be constituted by the red interference pigment, when said pigment presents non-zero magnetic susceptibility.

In another of its aspects, the invention provides a cosmetic composition comprising, dispersed in a cosmetically acceptable medium:
an interference first pigment that is red and that, when the composition is applied to a surface, is capable of generating red highlights with an intensity of 3000 cd·m⁻² or more and with a dominant wavelength λ₁ in the range 580 nm to 650 nm; and
a reflective second pigment that is silvery or that is colored with a dominant wavelength λ₂ such that |λ₁−λ₂|≧50 nm, this second pigment having an average size that is 30 μm or more, better 40 μm.

The second pigment may be an interference pigment.

The applicant noted that the second pigment can bring new color effects while making it possible for the composition to preserve the intensity of brightness of the red interference pigment, the first and second pigments being able to create, to some extent, a coloured mosaic.

A difficulty can appear in the formulation of the composition when it is wanted to have intensities of highlights of the same order for the red interference pigment and the colored reflective pigments, in order to obtain an effect of relatively homogeneous pixellisation in intensity.

When the coloured reflective pigments have a multi-layer structure, it can be advantageous to use a red interference pigment and coloured reflective pigments having the same heart, because that can make it possible to more easily obtain the same surface quality, which strongly influences the intensity of highlights.

The use of the same heart can also make it possible to more easily obtain the same color generated by absorption when the red interference pigment and the colored reflective pigments present a surfacing carried out in same material, which can be interesting so that the red interference pigment and the colored reflective pigments appear with the same color under almost horizontal light.

In another one of its aspects, the invention provides a cosmetic composition comprising, in a cosmetically acceptable medium:
at least one red interference pigment that, when the composition is applied to a support, can generate highlights with an intensity of 3000 cd·m⁻² or more and a dominant wavelength in the range 580 nm to 680 nm; and
at least one coloring agent which is sensitive to at least one external stimulus.

The combined use of a red interference pigment and the Xchrome coloring agent can produce at least two different appearances for the composition depending on the state of the Xchrome coloring agent.

It may be particularly esthetically pleasing if, in one of its states, the Xchrome coloring agent takes on a red color since that can reduce the contrast of the red highlights and render them less visible. The change in state of the Xchrome coloring agent is thus accompanied by better perception of the red highlights and the observer may be surprised to see the interference pigment shine intensely.

Further, by changing state, the Xchrome coloring agent may influence the diffusion of light in the environment of the red interference pigment by acting as a color filter or locally as a secondary source of illumination.

In one example of the invention, the Xchrome coloring agent may be selected so that it takes at least two states in which the interference phenomenon is and is not affected or in which it is affected to different degrees.

The coloring agent that is sensitive to an external stimulus may be in solution in the medium, which may apply with a solvatochromic agent, for example. This may avoid diffusion of light by the Xchrome agent and weaken the interference phenomenon.

It may be particularly advantageous for the red interference pigment to have a dimension in the range 30 μm [micrometer] to 80 μm, i.e. substantially of the same order as the separating power of the eye, more preferably about 40 μm, and for the Xchrome coloring agent to take on a red color in one of its states. Thus, a matte red background is obtained with highlights that appear to scintillate because of their particular dimensions, creating a sparkling effect.

In another one of its aspects, the invention provides a set comprising:
a first cosmetic composition for applying to keratinous substances, and comprising at least a diffusing filler or a coloring agent that is capable of generating a color by absorption, and
a second cosmetic composition for applying on the first and comprising a cosmetically acceptable medium in which there is dispersed at least one red interference pigment that, when the second composition is applied to a surface, is capable of creating highlights with an intensity of 3000 cd·m⁻² or more and with a dominant wavelength in the range 580 nm to 650 nm.

By means of this aspect of the invention, the interference phenomenon is not hampered by the presence of the diffusing pigment or of the filler since said pigment or said filler is present in the underlying base layer and consequently does not deleteriously affect the propagation of light in the covering layer containing the red interference pigment.

The medium in which the red interference pigment is dispersed is preferably transparent, thereby making it possible to see the underlying deposit.

In another one of its aspects, the invention also provides a set comprising:
a base composition comprising a cosmetically acceptable medium in which there is dispersed at least one red interference pigment that, when the composition is applied to a surface, is capable of creating highlights with an intensity of 3000 cd·m⁻² or more and with a dominant wavelength in the range 580 nm to 650 nm,
a covering composition for applying on the base composition. This other composition may be transparent and may serve, for example, to improve glossiness and create a magnifying-glass effect on the red highlight points.

The covering composition may comprise a medium having a refractive index that is greater than the refractive index of the medium in which the red interference pigment is dispersed.

The first composition may be for forming the base layer and may present any formulation that is compatible with subsequently depositing the second composition.

In particular, the first composition may comprise a cosmetically acceptable medium, as defined above, and at least one coloring agent or a diffusing filler.

The second composition contains the red interference pigment, dispersed in a cosmetically acceptable medium. The second composition is for applying on the first, for example.

Dark Skin

AS used in the context of the present invention, the term “dark skin” means skin having an average lightness L* measure on the forehead, the cheekbones, and the chin that is less than 55 in the CIE 1976 color space.

By way of example, the saturation C* may be in the range 10 to 30, in particular in the range 12 to 28. The hue-angle values h in ° may be in the range about 38° to about 54°, for example. The lightness values L* may be 50 or less, or even 45 or 40 for darker skin, while usually remaining greater than 30 for most skin colors.

By way of example, dark skin is found amongst African, African-American, Spanish-American, Indian, and North African populations.

Such skin can also be classified on the basis of its reactivity to the effects of solar radiation on the scale proposed by FITZPTRICK.

In that scale, the various existing ethnic skin types can be distinguished as follows:

| Type | Reactivity of the skin | Origin |
|---|---|---|
| I | Always burns, never tans | Celtic |
| II | Always burns, tans a little | Germanic |
| III | Burns moderately, tans | European |
| | progressively | |
| IV | Burns little, tans very easily | Mediterranean |
| V | Rarely burns, tans intensively | Middle Eastern - |
| | | South American |
| VI | Never burns, highly pigmented | African |

The dark skin to which the present invention relates more particularly comes within types IV to VI.

Saturation Measurement

The composition was spread onto a contrast card of trade name LENETA with a thickness of 300 μm. The measurement was taken on the black background of the card.

Reflectance was measured using a Minolta 3700-d (d65/10°) spectrocolorimeter in “specular component excluded” mode, with a small aperture (CREISS).

The reflectance spectra obtained were expressed in colorimetric coordinates in the Commission Internationale de l'Eclairage's CIELab76 color space, in accordance with recommendation 15:2004.

Measurement of Intensity of Highlights

To measure the intensity of the highlights, the study composition was spread onto a contrast card, for example from LENETA, at a thickness of 300 μm.

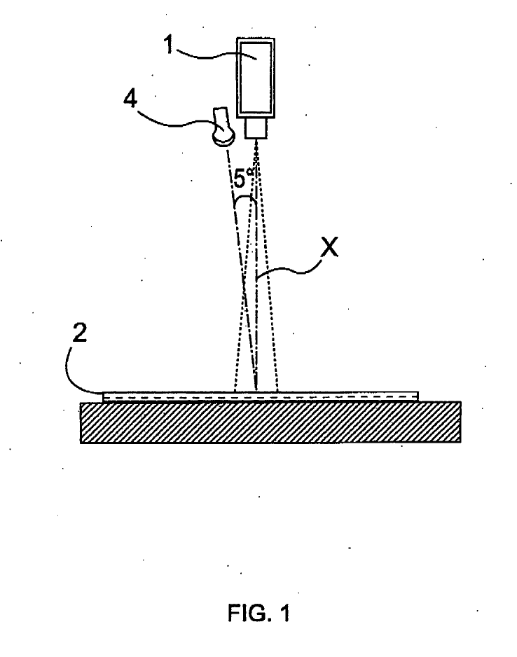

The spread composition was placed in front of a calorimetric camera 1 in the arrangement shown in FIG. 1. In this figure, the contrast card 2 coated with the composition was placed perpendicular to the optical axis X of the camera 1 and illuminated by means of a light source 4 (D65 illuminant) emitting in a direction making an angle of 5° with the optical axis X.

The highlight is defined as the intensity of the locally emitted light.

The camera has a resolution of a few micrometers in the xy plane, sufficient to distinguish very clearly between the various particles present in the composition.

As an example, the optical system is the LUMICAM 1300 photometer and imaging calorimeter from INSTRUMENT SYSTEMS.

Luminance measurements can be made in the range 0.2 cd·m⁻² to 200000 cd·m⁻² with measurement accuracy of 4%, repeatability of 0.1%, and uniformity of 1.5% (for a 10×10 pixel zone).

The optical system comprises a 105 mm macro objective with a field angle of 5° and a focal length of 22 mm, placed at a distance of 48 cm [centimeter] from the composition. The measurement zone extends over 2.9×2.7 mm.

The sensitivity is 100 iso; the shutter speed is 1/60 s [second] and the aperture is f:2.

The experimental device shown can eliminate specular reflection over the surface of the composition film.

The result obtained is in the form of a two dimensional matrix where each element M_{i,j} represents the intensity detected by the cell with coordinates i,j in the xy plane, in candela per m²:

$$[ \begin{matrix}{M}_{1 , 1} & & … & & {M}_{1 , m} \\ & ⋰ & & ⋱ & \\ ⋮ & & {M}_{i , j} & & ⋮ \\ & ⋱ & & ⋰ & \\ {M}_{n , 1} & & … & & {M}_{m , n}\end{matrix} ]$$

where

m designates the number of pixels in the x direction of the detection system; and

n designates the number of pixels in the y direction of the detection system.

The dominant wavelength is measured with the imaging calorimeter.

Turbidity Measurement

Turbidity corresponds to the reduction in the transparency of a liquid as a result of the presence of particles in suspension, and is measured by passing a light beam through the sample being tested.

Turbidity can depend on the refractive index of the medium and on the kind and the concentration of bodies in suspension in said medium.

The turbidity index is determined by measuring the light that is diffused by the praticles in suspension, by means of a tubidimeter, in this event the turbidimeter referenced 2100 P by HACH.

Measurement of the Color Path

When the composition presents a turbidity index of 100 nephelometric turbidity units (NTU) or less, it makes it possible to obtain a relatively long color path, since the small total amount of particles in suspension does not hamper observation of the color produced by absorption by the surface layer of the high-index red interference pigment.

The term “color path” denotes a variation in the a*b* plane of the CIE 1976 calorimetric space and can, for example, be measured by means of a spectrogonioreflectometer of trade name INSTRUMENT SYSTEMS and of reference GON 360 GONIOMETER after the composition has been spread in the fluid state to a thickness of 300 μm by means of an automatic spreader onto a contrast card of trade name ERICHSEN and of reference Typ 24/5, the measurement being taken on the black background of the card.

The color path of a composition of the invention corresponds to a variation Dh in the hue angle h of at least 20°, for example, when the observation angle is varied in the range 0 to 800 relative to the normal, for a light at an angle of incidence of 45°.

Red Interference Pigment

In accordance with the invention, this pigment can generate highlights with a dominant wavelength in the range 580 nm to 650 nm, preferably 580 nm to 600 nm, with an intensity of 3000 cd·m⁻² or more, preferably 3400 cd·m⁻², more preferably 4200 cd·m⁻². The intensity may be less than 5000 cd·m⁻².

By way of example, the amount of red interference pigment may lie in the range 0.1% to 15% when the medium is liquid, and may lie in the range 0.1% to 60% when the medium is solid.

Preferably, the dimension of said pigment, defined by the mean granulometric distribution at the population mid point, also denoted D₅₀, is 30 μm or more, preferably 40 μm, for example in the range 30 μm to 80 μm, more preferably 30 μm to 70 μm.

The pigment is advantageously generally flattened in shape, its thickness being 5 μm or less, for example, preferably 3 μm or less.

The multi-layered structure may optionally be symmetrical, and is preferably symmetrical.

The pigment may comprise a core of an organic or inorganic material, covered with one or more layers of organic or inorganic materials.

The pigment may, for example, comprise a core of silica, mica, or glass coated with a layer of iron oxide Fe₂O₃ or another metal oxide, for example a titanium or tin oxide.

The thickness of the layer or layers covering the core is determined by the theory of the reflection of light on thin layers so that the reflected light has the desired dominant wavelength.

Preferably, the core is generally flattened in shape and the pigment has substantially flat principal faces to allow intense specular reflection.

If appropriate, the pigment may have a non-zero magnetic susceptibility.

An example of a commercially available red interference pigment that can be mentioned is that sold with the reference XIRONA LE ROUGE by MERCK.

Cosmetically Acceptable Medium

The term “cosmetically acceptable medium” denotes a non-toxic medium which is capable of being applied to the keratinous substances of human beings.

The cosmetically acceptable medium should be adapted to the nature of the surface on which the composition is to be applied and to the form in which the composition is intended to be packaged.

The composition of the invention may comprise an aqueous medium and/or a fatty phase.

Aqueous or Fatty Phase

The composition may comprise water or a mixture of water and hydrophilic organic solvents such as alcohols, in particular linear or branched lower mono-alcohols containing 2 to 5 carbon atoms, such as ethanol, isopropanol or n-propanol, polyols such as glycerin, diglycerin, propylene glycol, sorbitol, penthylene glycol, or polyethylene glycols.

The hydrophilic phase may also contain C₂ ethers and C₂-C₄ hydrophilic aldehydes.

Water or a mixture of water and hydrophilic organic solvents may be present in the composition of the invention in an amount of 0 to 90%, in particular 0.1% to 90% by weight relative to the total composition weight, preferably 0 to 60% by weight, in particular 0.1% to 60% by weight.

The composition may also include a fatty phase, in particular constituted by fats which are liquid at 25° C. and optionally fats which are solid at ambient temperature, such as waxes, pasty fats, gums, and mixtures thereof.

Fats that are liquid at ambient temperature, usually termed “oils”, that can be used in the invention and can be mentioned amongst others are: hydrocarbon-containing vegetable oils such as liquid triglycerides of fatty acids containing 4 to 10 carbon atoms, for example heptanoic or octanoic acid triglycerides, or sunflower, corn, soya, grapeseed, sesame seed, apricot kernel, macadamia nut, castor, or avocado stone oil, caprylic/capric acid triglycerides, jojoba oil, shea nut butter oil; linear or branched hydrocarbons of mineral or synthetic origin, such as paraffin oils, in particular C₈-C₁₆ isoparaffins such as isododecane, isodecane, isohexadecane, Vaseline, polydecenes, hydrogenated polyisobutene such as Parleam®, squalane, synthesized esters and ethers, in particular fatty acids such as Purcellin oil, isopropyl myristate, 2-ethylhexyl palmitate, 2-octyldodecyl stearate, 2-octyldodecyl erucate, isostearyl isostearate; hydroxylated esters such as isostearyl lactate, octylhydroxystearate, octyldodecyl hydroxystearate, diisostearylmalate, triisocetyl citrate, fatty alcohol heptanoates, octanoates or decanoates; polyol esters such as propylene glycol dioctanoate, neopentylglycol diheptanoate, diethyleneglycol diisononanoate; and pentaerythritol esters; fatty alcohols containing 12 to 26 carbon atoms, such as octyldodecanol, 2-butyloctanol, 2-hexyldecanol, 2-undecylpentadecanol or oleic alcohol; partially hydrocarbonated and/or siliconized fluorinated oils; silicone oils such as volatile or non volatile, linear or cyclic polymethylsiloxanes (PDMS) which may be liquid or pasty at ambient temperature, such as cyclomethicones or dimethicones, optionally comprising a phenyl group, such as phenyl trimethicones, phenyltrimethylsiloxydiphenyl siloxanes, diphenylmethyldimethyl-trisiloxanes, diphenyl dimethicones, phenyl dimethicones, polymethylphenylsiloxanes; and mixtures thereof.

Said oils may be present in an amount of 0.01% to 90% relative to the total composition weight.

The composition of the invention may also include one or more organic solvents that are physiologically acceptable. The solvent or solvents, which may be lipophilic, may be present in an amount of 0 to 90%, preferably 0 to 60% by weight relative to the total composition weight, more preferably 0.1% to 30%.

The medium may include a liquid organic phase in which the water is dispersed or emulsified.

The composition may also have a continuous fatty phase, which may contain less than 5% water, in particular less than 1% water relative to the total weight and in particular it may be in the anhydrous form.

Film-Forming Agent

The medium may include a film-forming agent, in particular a film-forming polymer.

The term “film-forming agent” means an agent which can, by itself or in the presence of an auxiliary film-forming agent, form a macroscopically continuous film that adheres to keratinous substances, preferably a cohesive film and more preferably a film the cohesion and mechanical properties of which are such that said film may be isolated and manipulated in isolation, for example when said film is produced by casting onto a non-stick surface such as a Teflon or silicone surface.

The composition may comprise an aqueous phase and the film-forming polymer may be present in this aqueous phase. It may be a polymer in dispersion or in solution.

The composition may include an oily phase and the film-forming polymer may be present in said oily phase. The polymer may then be in dispersion or in solution.

Examples of film-forming polymers that can be used and that can be mentioned are synthetic polymers, of the radical or polycondensate type, polymers of natural origin, and mixtures thereof.

The radical type film-forming polymers may in particular be vinyl polymers or copolymers, in particular acrylic polymers.

Film-forming polycondensates that can be mentioned include polyurethanes, polyesters, polyester amides, polyamides, epoxyester resins, and polyureas.

The polyesters may be obtained, in known manner, by polycondensation of dibasic carboxylic acids with polyols, in particular diols.

The polyester amides may be obtained in a manner analogous to the polyesters, by polycondensation of dibasic acids with diamines or amino alcohols.

Examples of liposoluble film-forming polymers that can be mentioned are vinyl ester copolymers (the vinyl group being directly bonded to the oxygen atom of the ester group and the vinyl ester having a saturated, linear or branched hydrocarbon radical containing 1 to 19 carbon atoms, bonded to the carbonyl group of the ester group) and at least one other monomer which may be a vinyl ester (different from the vinyl ester already present), an α-olefin (containing 8 to 28 carbon atoms), an alkylvinylether (the alkyl group of which contains 2 to 18 carbon atoms) or an allyl or methallyl ester (containing a saturated, linear or branched hydrocarbon radical containing 1 to 19 carbon atoms, bonded to the ester group).

Said copolymers may be cross-linked using cross-linking agents which may either be of the vinyl type or of the allyl or methallyl type, such as tetraallyloxyethane, divinylbenzene, divinyl octanedioate, divinyl dodecanedioate or divinyl octadecanedioate.

Examples of such copolymers that can be mentioned are: vinyl acetate/allyl stearate, vinyl acetate/vinyl laurate, vinyl acetate/vinyl stearate, vinyl acetate/octadecene, vinyl acetate/octadecylvinylether, vinyl propionate/allyl laurate, vinyl propionate/vinyl laurate, vinyl stearate/1-octadecene, vinyl acetate/1-dodecene, vinyl stearate/ethylvinylether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl propionate/vinyl stearate, allyl dimethyl propionate/vinyl stearate, vinyl propionate/vinyl stearate, cross-linked with 0.2% of divinyl benzene, vinyl dimethyl propionate/vinyl laurate, cross-linked with 0.2% of divinyl benzene, vinyl acetate/octadecyl vinyl ether, cross-linked with 0.2% of tetraallyloxyethane, vinyl acetate/allyl stearate, cross-linked with 0.2% of divinyl benzene, vinyl acetate/octadecene-1 cross-linked with 0.2% of divinyl benzene and allyl propionate/allyl stearate cross-linked with 0.2% of divinyl benzene.

The film-forming polymer may also be selected from silicone resins, which are generally soluble or swellable in silicone oils which are cross-linked polyorganosiloxane polymers.

The film-forming polymer may also be present in the composition in the form of particles in dispersion in an aqueous phase or in a non-aqueous solvent phase, generally termed a latex or pseudolatex. Techniques for preparing said dispersions are well known to the skilled person.

The composition of the invention may include a plasticizing agent encouraging the formation of a film with the film-forming polymer. Such a plasticizing agent may be selected from all compounds known to the skilled person to be capable of carrying out the desired function.

Clearly, this list of polymers is not exhaustive.

Fillers and Other Coloring Agents

The cosmetic composition may include fillers, in particular colorless fillers, in the medium.

The term “fillers” means particles of any form which are insoluble in the medium of the composition, regardless of the temperature at which the composition is produced. Said fillers serve in particular to modify the rheology or texture of the composition.

Examples of fillers that can be mentioned amongst others are talc, mica, silica, kaolin, and polyamide powders (Nylon® or Orgasol from Atochem).

In some embodiments of the invention, the fillers can be white or colorless in the medium. Colorless fillers are preferably used in the medium rather than white fillers in the medium.

Examples of colorless fillers in the medium that can be mentioned amongst others are mica, and thermoplastic material powders, polyamide powders (e.g. Nylon® or Orgasol from Atochem), polyethylene terephthalate (PET), polyethylene (PE), polypropylene (PP), polyvinyl chloride (PVC), polymethyl methacrylate (PMMA), polycarbonate (PC) powders.

Examples of white fillers in the medium that can be mentioned amongst others are talc titanium dioxide, barium sulfate, kaolin, silica, and magnesium sulfate.

The amount of filler should be selected so that it does not in any way deleteriously affect the interference phenomenon responsible for the red highlights.

The composition may include a coloring agent other than the red interference pigment, in an amount that does not prevent the interference phenomenon that produces the red highlights.

This coloring agent may be a diffusing pigment that is capable of generating a color by an absorption phenomenon and that comprises a substance that is organic or inorganic.

The coloring agent may also include a diffractive pigment, a nacre, or a goniochromatic pigment.

The coloring agent may also include a colorant.

It may be a colorant of animal, vegetable, or mineral origin, in particular of vegetable or mineral origin, especially of vegetable origin. Said colorant may be non synthetic in nature.

The colorant may be a hydrosoluble or liposoluble natural colorant.

Particular illustrative examples of natural hydrosoluble coloring agents that can be used in the context of the invention that can be mentioned are caramel, beetroot juice and carmine, betanin (beetroot), copper chlorophyllin, methylene blue, anthocyanins (enocianin, black carrot, hibiscus, elder), and riboflavin.

Particular illustrative examples of natural liposoluble coloring agents which may be used in the context of the invention that can be mentioned are Sudan red, β-carotene, carotenoids, lycopene, palm oil, Sudan brown, quinoline yellow and xanthophylles (capsanthin, capsorubin, lutein), and curcumin.

Other natural colorants that can be mentioned in particular are anthocyans from flowers or fruit and their derivatives, flavonoids and tannins extracted from native or fermented vegetables, juglone, lawsone, fermented soya extracts, algae, fungi, micro-organisms, Flavylium salts not substituted in the 3-position as described in European patent EP-A-1 172 091, extracts from Gesneria Fulgens, Blechum Procerum, Saxifraga and pigments which can be obtained by extraction with an organic or hydro-organic solvent from a culture medium of micromycetes of the monascus Monascus type.

Examples of synthetic colorants that can be mentioned are synthetic liposoluble colorants such as, for example, DC Red 17, DC Red 21, DC Red 27, DC Green 6, DC Yellow 11, DC Violet 2, and DC Orange 5.

Examples of synthetic hydrosoluble colorants that can be mentioned are FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, and FDC Blue 1.

Reflective Particles

Various reflective particles having a metallic glint can be envisaged provided they present reflectivity that is high enough to create highlights with an intensity that is greater than or equal to 3000 cd·m⁻², better 4 000 cd m⁻², and for example less than or equal to 5 000 cd m².

The ratio m₁/m₂ of the weight m₁ of red interference pigment over the weight m₂ of reflective particles can lie in the range 0.1 to 1.5.

Their size can lie in the range 10 μm to 500 μm, for example, preferably lying in the range 10 μm to 150 μm. The size can advantageously be greater than or equal to 40 μm.

The reflective particles can be in the form of flakes, thereby making the reflection more directional, or, in contrast, they can present a substantially spherical shape, in order to provide reflection that is more diffuse.

By way of example, the reflective particles have a metallic glint, and they advantageously include at least one electrically-conductive surface layer that is formed by at least one metal or metal oxide.

Regardless of their form, the reflective particles having a metallic glint may optionally have a multilayer structure; with a multilayer structure, they may, for example, have at least one layer preferably having uniform thickness, in particular of a reflective material, advantageously a metal compound.

When the reflective particles having a metallic glint do not have a multilayer structure, they may, for example, be composed of at least one metal compound, e.g. a metal oxide, in particular an iron oxide obtained by synthesis.

When the reflective particles have a multilayer structure they may, for example, comprise a natural or synthetic substrate, in particular a synthetic substrate which is at least partially coated with at least one layer of a reflective material, in particular at least one layer of at least one metal compound such as a metal or an alloy. The substrate may be a single material or multiple materials, and it may be organic and/or inorganic. More particularly, the substrate may be selected from glasses, ceramics, graphite, metal oxides, aluminas, silicas, silicates, in particular aluminosilicates and borosilicates, synthetic mica, and mixtures thereof, this list not being limiting.

Examples of reflective particles comprising a mineral substrate coated with a metal layer that may be mentioned are particles comprising a substrate of borosilicate coated with silver. Glass substrate particles coated with silver in the form of flakes are sold under the trade name MICROGLASS METASHINE REFSX 2025 PS by TOYAL. Glass substrate particles coated with nickel/chromium/molybdenum alloy are sold under the trade name CRYSTAL STAR GF 550, GF 2525 by the same company.

Regardless of their form, the reflective particles having a metallic glint may also be selected from particles of synthetic substrate at least partially coated with at least one layer of at least one metal oxide selected, for example, from oxides of titanium, in particular TiO₂, of iron, in particular Fe₂O₃, of tin, or of chromium, barium sulfate, and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and their mixtures or alloys.

Examples of such particles that may be mentioned are particles comprising a substrate of synthetic mica coated with titanium dioxide, or glass particles coated either with brown iron oxide, titanium oxide, tin oxide, or one of their mixtures such as those sold under the trade name REFLECKS® by ENGELHARD.

Other examples of reflective particles having a metallic glint, presenting a metal compound at their surface or including at least one coated metal compound, and that may be mentioned are the particles proposed under the trade name METASHINE® ME 2040 PS, METASHINE® MC5090 PS, or METASHINE® MC280GP (2523) by NIPPON SHEET GLASS, SPHERICAL SILVER POWDER® DC 100, SILVER FLAKE® JV6, or GOLD POWDER® A1570 by ENGELHARD, STARLIGHT REFLECTIONS FXM® by ENERGY STRATEGY ASSOClATES INC. BRIGHT SILVER® 1 E 0.008X0.008 by MEADOWBROOK INVENTIONS, ULTRAMIN® (ALUMINIUM POUDRE FINE LIVING), and COSMETIC METALLIC POWDER VISIONAIRE BRIGHT SILVER SEA®, COSMETIC METALLIC POWDER VISIONAIRE NATURAL GOLD® (60314), or COSMETIC METALLIC POWDER VISIONAIRE HONEY® (60316) by ECKART.

The reflective particles having a metallic glint may reflect the visible spectrum in substantially uniform manner, e.g. as with particles that are optionally coated in a metal such as silver or aluminum, which can thus lead to a metallic glint having a non-neutral, yellow, pink, red, bronze, orange, brown, gold, and/or copper glint, depending on the kind of metal compound at the surface, for example.

The reflective particles having a metallic glint may be present in the composition in an amount in the range 0.1% to 60% by weight relative to the total weight of the first composition, specifically 1% to 30% by weight, e.g. 3% to 10% by weight.

When reflective particles have a multilayer structure with a core, the core can be in the same material as the core of the red interference pigment.

Silvery Reflective Pigments

This pigment reflects the incident light spectrum in substantially uniform manner.

Examples of silvery reflective pigments that may be mentioned are silvery reflective particles TIMICA SPARKLE 110P®, TIMICA SILKBLANC 110W®, FLAMENCO SUPERPEARL 120 C+®, TIMICA EXTRA LARGE SPARKLE 110S®, FLAMENCO PEARL 110C®, TIMICA PEARL WHITE 110 A®, TIMICA SILVER SPARKLE 5500/EP 94003®, FLAMENCO SATIN PEARL 3500® sold by ENGELHARD, silvery reflective particles NAILSYN PLATINUM 60®, XIRONA SILVER®, BIRON LF 2000® (ref 117077), TIMIRON SNOWFLAKE MP 99® (117470), LOW LUSTRE PIGMENT® (17399), TIMIRON DIAMOND CLUSTER MP 149® (17266), TIMIRON ULTRALUSTER MP 111® (117226), TIMIRON PEARL SHEEN MP 30® (17216), TIMIRON SUPER SILK MP 1005® (17203) sold by MERCK, silvery reflective particles PRESTIGE SPARKLING SILVER® (35178), PRESTIGE SPARKLING SILVER STAR® (35179) sold by ECKART, silvery reflective particles SUNSHINE FINE WHITE® (C80-3100), SHUNSHINE GLITTER WHITE® (C80-3400) sold by SUN, and silvery reflective particles KTZ CLASSIC WHITE® (10-40 MICRONS), KTZ STELLAR WHITE® (20-80 MICRONS) sold by TAIZHU.

Colored Reflective Pigments

Various colored reflective pigments other than the red interference pigment can be envisaged, provided they present reflectivity that is high enough to create highlights with an intensity that is greater than or equal to 3000 cd·m⁻², better 4 000 cd m⁻², and for example less than or equal to 5 000 cd m⁻².

Their size is preferably greater than or equal to 30 μm, better 40 μm advantageously being of the same order as the size of the red interference pigment, to within 10%, in order to obtain a pixellization effect that is more uniform. In particular, the size can lie in the range 30 μm to 80 μm, for example.

The colored reflective pigment can have a dominant wavelength that is different from the dominant wavelength of the red interference pigment, e.g. 580 nm or less, measured with the above-mentioned calorimeter, under the measurement conditions used for measuring the intensity of the highlights.

It can be advantageous for the colored reflective pigment to have a core of the same material as the red interference pigment, since that makes it possible to have highlight intensities of the same order, to within 10%.

The expression “of the same order, to within 10%” signifies that the size or the highlight intensity of the reflective pigment is in the range 0.9 to 1.1 times the size or the highlight intensity of the red interference pigment.

The surface layer of the colored reflective pigment can be of the same material as the surface layer of the red interference pigment, in particular when the core is also of the same material, the pigments thus differing by the thickness of the surface layer, for example, thereby making it possible to generate another color by the interference phenomenon.

By way of example, the proportion of colored reflective pigment lies in the range 0.1 to 10 times the proportion of the red interference pigment.

Proportions similar to within 10% make it possible to obtain a uniform effect.

The colored reflective pigments can be selected from goniochromatic nacres et interference pigments, amongst others.

The term “nacre” means colored particles of any form, which may optionally be iridescent, as produced in the shells of certain mollusks, or which are synthesized, and which exhibit a “pearlescent” coloring effect by an interference phenomenon.

Nacres may be selected from nacre pigments such as mica titanium coated with iron oxide, mica coated with bismuth oxychloride, mica titanium coated with chromium oxide, mica titanium coated with an organic colorant, in particular of the type mentioned above, and nacre pigments based on bismuth oxychloride. They may also be particles of mica on the surface of which at least two successive layers of metal oxides and/or organic coloring substances have been superimposed.

More particularly, the nacres may have a yellow, pink, red, bronze, orange, brown, gold, and/or coppery color or glint.

Illustrative examples of nacres suitable for being introduced into the composition and that may be mentioned are colored pigments TIMICA SPARKLE GOLD®, CLOISONNE SPARKLE ROUGE 450J®, FLAMENCO SPARKLE GOLD 220J®, FLAMENCO SPARKLE GREEN 820J®, FLAMENCO SPARKLE ORANGE 320J®, FLAMENCO SPARKLE BLUE 620J®, CLOISONNE SPARKLE GOLD 222J®, CLOISONNE SPARKLE GOLD 222J®, CLOISONNE SPARKLE BLUE-ROUGE 650J®, FLAMENCO SPARKLE VIOLET 520J®, CLOISONNE SPARKLE COPPER 350J®, CLOISONNE SPARKLE BRONZE 250J®, DUOCROME SPARKLE BY 226J®, DUOCROME SPARKLE RY 224J/EP 980010®, DUOCROME SPARKLE BR 426J®, DUOCROME SPARKLE RB 624J/EP 98002®, FLAMENCO SPARKLE RED 420J® sold by ENGELHARD, colored pigments TIMIRON DIAMOND CLUSTER MP 149 (17266)® sold by MERCK, and colored pigments KTZ ULTRA SHIMMER® sold by TAIZHU.

Active Ingredients and Other Compounds

The cosmetic composition may also contain one or more cosmetic, dermatological, sanitary or pharmaceutical active ingredients.

Examples of cosmetic, dermatological, sanitary or pharmaceutical active ingredients which may be used in the compositions of the invention that can be mentioned are moisturizers (polyols such as glycerin), vitamins (C, A, E, F, B or PP), essential fatty acids, essential oils, ceramides, sphingolipids, liposoluble sunscreens or sunscreens in the form of nano particles, specific active ingredients for the treatment of skin (protective agents, anti-bacterial agents, anti-wrinkle agents, etc). Said active ingredients may, for example, be used in concentrations of 0.001% to 15% relative to the total composition weight.

The cosmetic composition may also contain ingredients which are routinely used in cosmetics, such as thickeners, surfactants, oligo-elements, moisturizers, softening agents, sequestrating agents, fragrances, alkalinizing or acidifying agents, preservatives, antioxidants, UV screens, or mixtures thereof.

Depending on the type of application envisaged, the cosmetic composition may also include constituents conventionally used in the fields under consideration, which are present in a quantity appropriate to the desired galenical form.

Volatile Solvent

The composition may include at least one aqueous or organic solvent, in particular a volatile organic solvent.

The term “volatile solvent” as used in the context of the present invention means a solvent that is liquid at ambient temperature, having a non-zero vapor pressure at ambient temperature and atmospheric pressure, in particular a vapor pressure in the range 0.13 pascals (Pa) to 40000 Pa (10⁻³ millimeters of mercury (mm Hg) to 300 mm Hg), and preferably in the range 1.3 Pa to 13000 Pa (0.01 mm Hg to 100 mm Hg), and preferably in the range 1.3 Pa to 1300 Pa (0.01 mm Hg to 10 mm Hg).

The first composition may include at least one volatile solvent constituted by a volatile oil.

The oil may be a siliconized oil or a hydrocarbonated oil, or may include a mixture of such oils.

The term “siliconized oil” as used in the context of the present invention means an oil including at least one silicon atom, and in particular at least one Si—O group.

The term “hydrocarbonated oil” means an oil containing mainly hydrogen and carbon atoms and possibly oxygen, nitrogen, sulfur, and/or phosphorus atoms.

The volatile hydrocarbonated oils may be selected from hydrocarbonated oils having 8 to 16 carbon atoms, and in particular C₈-C₁₆ branched alkanes (also termed isoparaffins) such as isododecane (also termed 2,2,4,4,6-pentamethylheptane), isodecane, isohexadecane, and oils sold under the trade names Isopars® or Permethyls®, for example.

Volatile oils that may also be used are volatile silicones, such as volatile linear or cyclic silicone oils, for example, in particular oils having a viscosity ≦8 centistokes (cSt) (8×10⁻⁶ square meters per second (m²/s)), and having in particular 2 to 10 silicon atoms, and in particular 2 to 7 silicon atoms, the silicones possibly including alkyl or alkoxy groups having 1 to 10 carbon atoms. In the invention, suitable volatile silicone oils that may be mentioned are in particular dimethicones having a viscosity of 5 cSt to 6 cSt, octa-methylcyclotetrasiloxane, decamethylcyclopentasiloxane, dodecamethylcyclohexasiloxane, heptamethylhexyltri-siloxane, heptamethyloctyltrisiloxane, hexamethyldi-siloxane, octamethyltrisiloxane, decamethyltetrasiloxane, dodecamethylpentasiloxane, and mixtures thereof.

Mention may also be made of volatile alkyltrisiloxane linear oils of general formula (I):

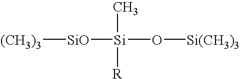

in which R represents an alkyl group having 2 to 4 carbon atoms and in which one or more hydrogen atoms can be substituted by an atom of fluorine or chlorine.

Oils of general formula (I) that may be mentioned are:
3-butyl 1,1,1,3,5,5,5-heptamethyl trisiloxane,
3-propyl 1,1,1,3,5,5,5-heptamethyl trisiloxane, and
3-ethyl 1,1,1,3,5,5,5-heptamethyl trisiloxane,

corresponding to oil of formula (I) in which R is respectively a butyl group, a propyl group, or an ethyl group.

It is also possible to use fluorinated volatile oils such as nonafluoromethoxybutane or perfluoromethyl-cyclopentane, and mixtures thereof.

By way of example, a composition of the invention may contain 0.01% to 95% by weight of volatile oil relative to the total weight of the composition.

The composition may comprise at least one organic solvent selected from the following list:
ketones that are liquid at ambient temperature, such as methylethylketone, methylisobutylketone, diisobutylketone, isophorone, cyclohexanone, or acetone;
alcohols that are liquid at ambient temperature, such as ethanol, isopropanol, diacetone alcohol, 2-butoxyethanol, or cyclohexanol;
glycols that are liquid at ambient temperature, such as ethylene glycol, propylene glycol, pentylene glycol, or glycerol;
propylene glycol ethers that are liquid at ambient temperature, such as propylene glycol monomethyl ether, the acetate of propylene glycol monomethyl ether, or dipropylene glycol mono n-butyl ether;
short-chain esters (containing a total of 3 to 8 carbon atoms), such as ethyl acetate, methyl acetate, propyl acetate, n-butyl acetate, or isopentyl acetate; and
alkanes that are liquid at ambient temperature, such as decane, heptane, dodecane, or cyclohexane.

The composition may also comprise water or a mixture of water and hydrophilic organic solvents which are routinely used in cosmetics, such as alcohols, in particular linear or branched lower monoalcohols containing 2 to 5 carbon atoms, such as ethanol, isopropanol or n-propanol, polyols such as glycerine, diglycerine, propylene glycol, sorbitol, penthylene glycol, or polyethylene glycols. The composition may also contain hydrophilic C₂ ethers and C₂-C₄ aldehydes. The water or mixture of water and hydrophilic organic solvents may be present in the composition in an amount in the range 0% to 90%, in particular 0.1% to 90% by weight, and preferably 0% to 60% by weight, more particularly 0.1% to 60% by weight relative to the total weight of the composition.

Magnetic Bodies

The expression “magnetic bodies” should not be understood in limiting manner and covers particles, fibers, clumps of particles and/or fibers, of any form, presenting non-zero magnetic susceptibility.

The concentration of magnetic bodies in the composition is selected in such a manner as to enable the interference phenomenon to appear in order to create red highlights. The concentration lies in the range about 0.05% to about 50% by weight, for example, in particular in the range about 0.1% to about 40% by weight, better in the range about 1% to about 30% by weight, depending on the kind of magnetic bodies and their incidence on the diffusion of light.

The applied composition may include magnetic fibers or other aspherical bodies, such as chains of particles or of fibers.

In the absence of a magnetic field, the magnetic bodies preferably do not present any remanent magnetism.

The magnetic magnetic bodies may comprise any magnetic material that presents sensitivity to the lines of a magnetic field, regardless of whether the field is produced by a permanent magnet or is the result of induction, the material being selected from nickel, cobalt, iron, and alloys and oxides thereof, in particular Fe₃O₄, and also from gadolinium, terbium, dysprosium, erbium, and alloys and oxides thereof, for example. The magnetic material may be of the “soft” or of the “hard” type. In particular, the magnetic material may be soft iron.

The magnetic bodies may optionally present a multilayer structure including at least one layer of a magnetic material such as iron, nickel, cobalt, and alloys and oxides thereof, in particular Fe₃O₄, for example.

The magnetic bodies are preferably aspherical, presenting an elongate shape, for example. Thus, when the bodies are subjected to the magnetic field, they tend to become oriented with their longitudinal axes in alignment with the field lines, and they are subjected to a change in orientation which results in the composition changing in appearance.

When the magnetic bodies are particles that are substantially spherical, their appearance is preferably non-uniform, so that a change in orientation results in a change in appearance.

Regardless of their shape, the size of the bodies may be in the range 1 nanometer (nm) to 10 millimeters (mm), for example, preferably in the range 10 nm to 5 mm, and more preferably in the range 100 nm to 1 mm, e.g. in the range 0.5 μm to 300 μm or 1 μm to 150 μm.

When the bodies are particles that do not have an elongate shape or that have an elongate shape with a form factor that is fairly small, the size of the particles if less than 1 mm, for example.

The magnetic bodies are magnetic pigments, for example.

Magnetic Pigments

Particularly suitable pigments are nacres comprising iron oxide Fe₃O₄. By way of example, pigments presenting magnetic properties are those sold under the trade names COLORONA BLACKSTAR BLUE, COLORONA BLACKSTAR GREEN, COLORONA BLACKSTAR GOLD, COLORONA BLACKSTAR RED, CLOISONNE NU ANTIQUE SUPER GREEN, MICRONA MATTE BLACK (17437), MICA BLACK (17260), COLORONA PATINA SILVER (17289), and COLORONA PATINA GOLD (117288) by MERCK, or indeed FLAMENCO TWILIGHT RED, FLAMENCO TWILIGHT GREEN, FLAMENCO TWILIGHT GOLD, FLAMENCO TWILIGHT BLUE, TIMICA NU ANTIQUE SILVER 110 AB, TIMICA NU ANTIQUE GOLD 212 GB, TIMICA NU-ANTIQUE COPPER 340 AB, TIMICA NU ANTIQUE BRONZE 240 AB, CLOISONNE NU ANTIQUE GREEN 828 CB, CLOISONNE NU ANTIQUE BLUE 626 CB, GEMTONE MOONSTONE G 004, CLOISONNE NU ANTIQUE RED 424 CHROMA-LITE, BLACK (4498), CLOISONNE NU ANTIQUE ROUGE FLAMBE (code 440 XB), CLOISONNE NU ANTIQUE BRONZE (240 XB), CLOISONNE NU ANTIQUE GOLD (222 CB), and CLOISONNE NU ANTIQUE COPPER (340 XB) by ENGELHARD.

Examples of magnetic pigment suitable for entering into the formulation of the composition that may also be mentioned are black iron oxide particles, e.g. those sold under the trade name SICOVIT noir E172 by BASF.

The magnetic pigments may also comprise metallic iron, in particular passivated soft iron, e.g. obtained from carbonyl iron by implementing the method described in U.S. Pat. No. 6,589,331, the content of which is incorporated herein by reference. The particles may include an oxide surface layer.

The magnetic bodies may be in the form of flakes.

The size of the magnetic bodies may be less than or equal to 10 μm, or even 1 μm.

The size of the magnetic bodies may also lie in the range 30 μm to 80 μm, thereby making it possible to obtain a pixellization effect that is variable under the effect of the magnetic field, when the red interference pigment presents a size of the same order.

Magnetic Fibers

The term “fibers” means generally elongate bodies presenting, for example, a form factor in the range 3.5 to 2500 or 5 to 500, e.g. 5 to 150. The form factor is defined by the ratio L/D, where L is the length of the fiber and D is the diameter of the circle in which the widest cross-section of the fiber is inscribed.

By way of example, the cross-section of the fibers may be inscribed in a circle having a diameter in the range 2 nm to 500 μm, e.g. in the range 100 nm to 100 μm, or even 1 μm to 50 μm.

By way of example, the fibers may present a length in the range 1 μm to 10 millimeters (mm), e.g. 0.1 mm to 5 mm, or even 0.3 mm to 3.5 mm.

By way of example, the fibers may present a weight in the range 0.15 denier to 30 denier (weight in grams for 9 km of thread), e.g. 0.18 denier to 18 denier.

The cross-section of the fibers may be of any shape, e.g. circular, or polygonal, in particular square, hexagonal, or octagonal.

The composition may contain solid or hollow fibers that may be independent or interlinked, e.g. braided.

The composition may contain fibers having ends that are blunted and/or rounded, e.g. by polishing.

The shape of the fibers need not be significantly modified when they are inserted into the composition, with said fibers being initially rectilinear and sufficiently rigid to keep their shape. In a variant, the fibers may present flexibility that enables them to be substantially deformed within the composition.

The fibers may contain a non-zero amount, that may be as great as 100%, of a magnetic material selected from soft magnetic materials, hard magnetic materials, in particular based on iron, zinc, nickel, cobalt, or manganese, and alloys and oxides thereof, in particular Fe₃O₄, rare earths, barium sulfate, iron-silicon alloys, possibly containing molybdenum, Cu₂MnAl, MnBi, or a mixture thereof, this list not being limiting.

When the composition contains fibers containing magnetic particles, said magnetic particles may be present at least at the surface of the fibers, or only at the surface of the fibers, or only inside the fibers, or they may even be dispersed within the fibers in substantially uniform manner, for example.

By way of example, each fiber may include a non-magnetic core with a plurality of magnetic particles on its surface.

Each fiber may also include a synthetic matrix containing a plurality of magnetic grains dispersed therein.

Where appropriate, a synthetic material filled with magnetic particles may itself be covered by a non-magnetic membrane. By way of example, such a membrane constitutes a barrier isolating the magnetic material(s) from the surrounding environment and/or it can provide color. Each fiber may comprise a one-piece magnetic core and be covered by a non-magnetic membrane, or it may comprise a one-piece non-magnetic core and be covered by a magnetic membrane.

The composition may contain fibers made by extruding or co-extruding one or more polymeric materials, in particular thermoplastics and/or elastomers. One of the extruded materials may contain a filler of dispersed magnetic particles.

Each fiber may comprise a synthetic material selected from polyamides; polyethylene terephthalate (PET); acetates; polyolefins, in particular polyethylene (PE) or polypropylene (PP); polyvinyl chloride (PVC); polyester block amide; plasticized Rilsan®; elastomers, in particular polyester elastomers, polyethylene (PE) elastomers, silicone elastomers, nitrile elastomers; or a mixture of these materials, this list not being limiting.

The composition may contain composite fibers each comprising a magnetic core that is covered, at least in part, by at least one non-magnetic, synthetic, or natural material. By way of example, the magnetic core may be covered by co-extruding a membrane made of a non-magnetic material around the core.

The core may alternatively be covered in some other way, e.g. by polymerization in situ.

The core may be a single piece or it may include a filler of magnetic grains dispersed in a matrix.

The composition may also contain composite fibers obtained by covering a non-magnetic, synthetic, or natural core, with a synthetic material filled with magnetic particles, the core being composed of a fiber made of wood; rayon; polyamide; plant matter; or polyolefin, in particular polyethylene, Nylon®, polyimide-amide, or aramid, this list not being limiting.

The composition may also contain magnetic composite particles, in particular a magnetic latex.

Magnetic Composite Particles

A magnetic composite particle is a composite material constituted by an organic or an inorganic matrix and by magnetic grains. At their surfaces and/or within themselves, the magnetic composite particles may thus include grains of a magnetic material. The composite particles may be constituted by a magnetic core covered by an organic or an inorganic matrix, or they may be constituted by an organic or an inorganic core covered by a magnetic matrix.

The magnetic composite particles include one of the above-mentioned magnetic materials, for example.

The size of the magnetic composite particles may be in the range 1 nm to 1 mm, for example, preferably in the range 100 nm to 500 μm, and more preferably in the range 500 nm to 100 μm. The term “size” means the size given by the statistical grain size distribution at half the population, referred to as “D50”.

The thesis by C. GOUBAULT, dated Mar. 23, 2004, and incorporated herein by reference, refers, in chapter 1, to the prior art on the subject of magnetic composite particles, and draws up a list of preparation methods that are suitable for being used to prepare magnetic composite particles, namely separately synthesizing the magnetic grains and the matrix, synthesizing the magnetic grains in contact with the matrix, or synthesizing the matrix in the presence of the magnetic grains.

KISKER markets inorganic-matrix magnetic composite particles composed of silica. DYNAL, SERADYN, ESTAPOR, and ADEMTECH propose organic-matrix magnetic composite particles that are also suitable for being used in the invention.

More particularly, under the reference M1-070/60, ESTAPOR markets magnetic latex constituted by grains of ferrite that are evenly distributed in a polystyrene matrix, said latex including 65% iron oxide, the mean diameter of the polystyrene particles being 890 nm, and the dry material mass content being 10%.

Ferrofluid

The composition P may contain a ferrofluid, i.e. a stable colloidal suspension of magnetic particles, in particular of magnetic nanoparticles.

The particles, having a size of the order of several tens of nanometers, for example, are dispersed in a solvent (water, oil, organic solvent), either by means of a surfactant or a dispersant, or by electrostatic interactions.

By way of example, the ferrofluids can be prepared by grinding ferrites or other magnetic particles until nanoparticles are obtained, which particles are then dispersed in a fluid containing a surfactant which is absorbed by the particles and stabilizes them, or else they can be prepared by precipitating a metallic-ion solution in a basic medium.

Each particle of the ferrofluid presents a magnetic moment that is determined by the size of the particle, and by the nature of the magnetic material.

Under the action of a magnetic field, the magnetic moments of the particles tend to come into alignment with the field lines, with non-zero magnetization appearing in the liquid. If the field is removed, there is no hysteresis and magnetization drops to zero.

Beyond a field threshold value, it is also possible to cause macroscopic changes in the liquid, e.g. the appearance of peaks, or a change in Theological properties.

The term “ferrofluid” also encompasses an emulsion of ferrofluid droplets in a solvent. Each drop thus contains colloidal magnetic particles in stable suspension. This makes it possible to have a ferrofluid in any type of solvent. The size of the magnetic particles in suspension in the ferrofluid may be in the range 1 nm to 10 μm, for example, preferably in the range 1 nm to 1 μm, and more preferably in the range 1 nm to 100 nm. The term “size” means the size given by the statistical grain size distribution at half the population, referred to as “D50”.

Mention can be made in particular of ferrofluids sold by Liquids Research LTD under the references:

WHKS1S9 (A, B, or C), which is a water-based ferrofluid containing magnetite (Fe₃O₄), having particles of 10 nm in diameter.

WHJS1 (A, B, or C), which is an isoparaffin-based ferrofluid, containing magnetite (Fe₃O₄) particles that are 10 nm in diameter.

BKS25_dextran, which is a water-based ferrofluid stabilized by dextran, containing magnetite (Fe₃O₄) particles that are 9 nm in diameter.

Chains of Particles and/or of Magnetic Fibers

The composition may contain clumps of particles or fibers having a largest dimension, e.g. length, that may, for example, be in the range 1 nm to 10 mm, e.g. in the range 10 nm to 5 mm, or in the range 100 nm to 1 mm, or even in the range 0.5 μm to 3.5 mm, e.g. in the range 1 μm to 150 μm.

By way of example, chains of magnetic particles may be obtained by assembling colloidal magnetic particles, as described in the publications “Permanently linked monodisperse paramagnetic chains”, by E. M. Furst, C. Suzuki, M. Fermigier, A. P. Gast, Langmuir, 14, 7334-7336 (1998), “Suspensions of magnetic particles”, by M. Fermigier, Y. Grasselli, Bulletin of the SFP (105) July 1996, and “Flexible magnetic filaments as micromechanical sensors”, by C. Goubault, P. Jop, M. Fermigier, J. Baudry, E. Bertrand, J. Bibette, Phys. Rev. Lett., 91, 26, 260802-1 to 260802-4 (2003), the contents of which are incorporated herein by reference.

In particular, those articles describe how to proceed in order to obtain chains of magnetic-latex particles that include a polystyrene matrix containing grains of iron oxide with functions on the surface, and that are bonded together in permanent manner following a chemical reaction, in particular covalent bonds between the surfaces of adjacent particles; a method is also described of obtaining chains of ferrofluid-emulsion droplets that are bonded together by physical interactions. The length and the diameter of the permanent chains obtained in this way can be controlled. Such magnetic chains constitute anisotropic magnetic objects that can be oriented and displaced under the effect of a magnetic field.

The dimensions of the magnetic chains may satisfy the same conditions as for the magnetic fibers.

Xchrome Coloring Agent

As mentioned above, the Xchrome coloring agent may be selected so that it takes at least one state in which it generates a color that is red or close to that produced by interference by the red interference pigment or, in contrast, a different color.

The term “color that is close” means that the dominant wavelength is substantially the same, being in the range 580 nm to 650 nm, measured with the above-mentioned imaging calorimeter.

The Xchrome coloring agent may also be selected so that in one state it takes on a color close to that generated by absorption in the surface layer of the interference pigment. This allows the interference pigment to be embedded in the background color to draw an observer's attention to the red highlights when the state of the coloring agent changes.

They may be photochromic coloring agents.

Photochromic Coloring Agents

In general, a photochromic coloring agent is a coloring agent having the property of changing hue when it is illuminated or not illuminated by ultraviolet light and to re-establish its initial color when it is no longer illuminated or is illuminated by a light, or passes from a non-colored state to a colored state and vice versa. In other words, such an agent has different hues depending on whether it is illuminated with light containing a certain quantity of UV radiation.

In the presence of a low level of light, the photochromic coloring agent may take on a substantially non colored state, so that the intensity of the red highlights is not unduly attenuated by the photochromic coloring agent.

In the presence of strong illumination, the photochromic coloring agent may take on a colored state, for example a dark hue or a red color, attenuating the intensity of the red highlights, which may then appear less brilliant than in the presence of low level illumination. This effect may surprise the observer and render the makeup particularly attractive.

The photochromic coloring agent may have a difference ΔE of at least 5. ΔE designates the difference in hue observed in the photochromic substance between its excited state, i.e. in the presence of UV radiation, and its non-excited state, i.e. in the absence of UV radiation.

Reference may usefully be made to examples of photochromic agents described in United States patent application US-A-2004/0228818 the contents of which are hereby incorporated by reference, in particular those with a ΔE of more than 5, as determined using the test presented in this document.

Examples of photochromic coloring agents are naphthopyrane derivatives of the 2H-naphtho-[2,1-b]-pyrane type with formula (I) or 3H-naphtho-[2,1-b]-pyrane type with formula (II): 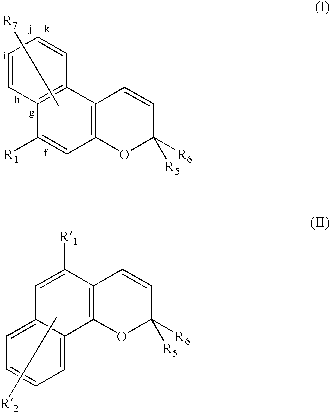
in which:

R₁ represents:

(i) a hydrogen atom;

(ii) a linear, branched, or cyclic, saturated or unsaturated hydrocarbon group containing 1 to 30 carbon atoms, optionally comprising 1 to 5 heteroatoms selected from N, O, S, Si, and P, and/or optionally halogenated or perhalogenated;

(iii) a hydrocarbon cycle formed with one of the “f” or “gh” bonds and the radical R₇; or

(iv) a group selected from —COOR₄, —C(O)NR₂R₃, —NR₂R₃, —OR₄ and —SR₄, in which:

R₂ and R₃ either independently represent a linear, branched, or cyclic, saturated or unsaturated hydrocarbon group containing 1 to 20 carbon atoms, optionally comprising 1 to 5 heteroatoms selected from N, O, S, Si, and P;

or, taken together with the nitrogen atom to which they are bonded, form a saturated or unsaturated hydrocarbon heterocycle containing 3 to 10 carbon atoms and optionally 1 to 5 other heteroatoms selected from N, O, S, Si and P, said cycle optionally being substituted with at least one linear, branched or cyclic, saturated or unsaturated hydrocarbon radical containing 1 to 20 carbon atoms optionally comprising 1 to 5 heteroatoms selected from N, O, S, Si, and P;

R₄ represents a linear, branched or cyclic, saturated or unsaturated hydrocarbon group containing 1 to 20 carbon atoms and/or optionally comprising 1 to 5 heteroatoms selected from N, O, S, Si, and P;

R₅ and R₆ independently represent a group selected from:

(i) saturated cyclic aminoaryl groups with formula (IIA) or (IIB): 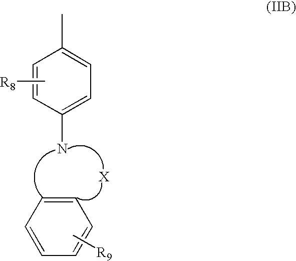
in which the cycle comprising N and X is a saturated cycle which contains a total of 3 to 30 atoms including nitrogen, the remainder being carbon atoms and/or heteroatoms selected from O, S, Si, P, and/or groups selected from —NH and —NR in which R represents a linear, branched, or cyclic, saturated or unsaturated hydrocarbon radical containing 1 to 20 carbon atoms, optionally comprising 1 to 5 heteroatoms selected from N, O, S, Si, and P;

(ii) indolinoaryl groups with formula (III): 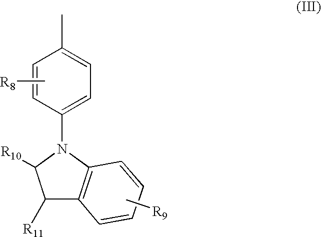
in which R₁₀ and R₁₁ independently represent a group selected from (i) linear, branched, or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated; (ii) halogen atoms; (iii) —CN (nitrile), —COOH (carboxylate), —NO₂ (nitro) groups; (iv) a hydrogen atom; (v) a group selected from —(O)NR₂R₃, —NR₂R₃, —OR₄ and —SR₄ in which R₂, R₃ and R₄ have the meanings given above; (vi) radicals R₁₀ and R₁₁ may together form a saturated or unsaturated hydrocarbon cycle having a total of 5 to 8 atoms (including the atoms of the indoline cycle), said atoms being selected from C, O, S and/or NR in which R represents H or a linear, branched or cyclic, saturated or unsaturated hydrocarbon radical containing 1 to 20 carbon atoms, optionally comprising 1 to 5 heteroatoms selected from N, O, S, Si, and P;

(iii) groups with formula (IV): 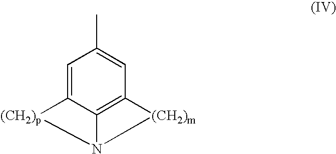
in which m and p are independently integers from 2 to 5;

(iv) unsaturated cyclic aminoaryl groups with formulae (VA), (VB), or (VC): 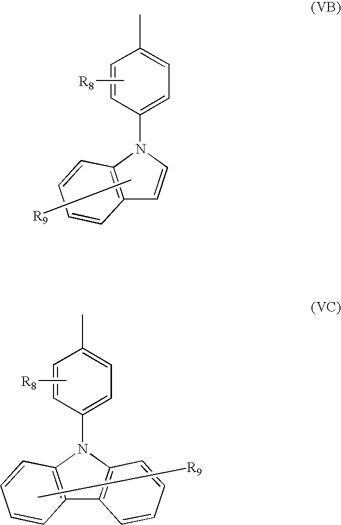
in which R₈ and R₉, independently represent a group selected from (i) linear, branched, or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated; (ii) halogen atoms; (iii) —CN (nitrile), —COOH (carboxylate), —NO₂ (nitro) groups; (iv) a hydrogen atom; (v) a group selected from —C(O)NR₂R₃, —NR₂R₃, —OR₄, and —SR₄, in which R₂, R₃ and R₄ have the meanings given above;

(v) a linear, branched or cyclic, saturated or unsaturated hydrocarbon group containing 1 to 30 carbon atoms optionally comprising 1 to 5 heteroatoms selected from N, O, S, Si and P; and in particular a group selected from —CONR₂R₃, —C₆H₄—NR₂R₃, and —C₆H₄—OR₄ in which R₂, R₃ and R₄ have the meanings given above;

R₇ represents a group selected from:

(i) linear, branched or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated;

(ii) halogen atoms;

(iii)-CN (nitrile), —COOH (carboxylate), —NO₂ (nitro); —N═N— (azo); ═NH (imino); —CONH₂ (amide) groups;

(iv) a hydrogen atom;

(v) a group selected from —C(O)NR₂R₃, —NR₂R₃, —OR₄ and —SR₄ in which R₂, R₃ and R₄ have the meanings given above;

(vi) radical R₇ may also form, with one of the “i”, “j”, “k”, or “g,h” bonds taken with radical R₁, or “f” taken with radical R₁, a saturated hydrocarbon cycle containing a total of 3 to 8 carbon atoms, optionally comprising 1 to 5 heteroatoms selected from N, O, S, Si, and P;

R′₁ represents a group selected from:

(i) a hydrogen atom;

(ii) a linear, branched or cyclic, saturated or unsaturated hydrocarbon group containing 1 to 30 carbon atoms optionally comprising 1 to 5 heteroatoms selected from N, O, S, Si, and P, and/or optionally halogenated or perhalogenated;

(iii) a group selected from —C(O)NR₂R₃, —NR₂R₃, —OR₄, and —SR₄, in which R₂, R₃ and R₄ have the meanings given above;

R′₂ represents a group selected from

(i) linear, branched or cyclic, saturated or unsaturated hydrocarbon groups containing 1 to 30 carbon atoms, optionally comprising 1 to 5 heteroatoms selected from N, O, S, Si and P, and/or optionally halogenated or perhalogenated;

(ii) halogen atoms;

(iii) —CN (nitrile), —COOH (carboxylate), —NO₂ (nitro); —N═N— (azo); ═NH (imino); —CONH₂ (amide) groups;

(iv) a hydrogen atom;

(v) a group selected from —C(O)NR₂R₃, —NR₂R₃, —OR₄ and —SR₄ in which R₂, R₃ and R₄ have the meanings given above.

Further examples of photochromic agents that may be mentioned are diarylethene with formula: 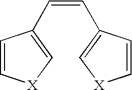
and its derivatives;

dihydroazulene/vinylhepta fulvene, with formula: 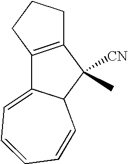
and its derivatives;

spyronaphthoxazine, with formula: 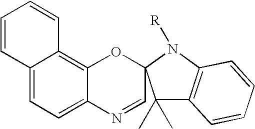
and its derivatives.

The photochromic agent may be an organic or an inorganic compound. When the photochromic agent is an organic compound, the color change may generally be more rapid and intense.

Examples of photochromic agents that may be mentioned are Photosol® from PPG, which reversibly changes color when activated by UV radiation with a wavelength in the range 300 nm to 360 nm, Reversacol® from J. ROBINSON and Photogenica® from CATALYST & CHEMICALS.

Thermochromic Agents

A thermochromic agent is a pigment or colorant that can change color as a function of temperature.

The thermochromic agent has, for example, a color that is lost when the temperature exceeds a certain value, for example about 15° C. or about 30° C., depending on the nature of the thermochromic agent.

The thermochromic agent may comprise capsules of a polymer containing a solvent, that solvent, depending on whether it is in the molten state or otherwise, allowing compounds to come into contact and modify the light absorption properties.

The color change may be reversible.

As an example, it is possible to use the thermochromic agent sold under the trade name Kromafast® Yellow5GX 02 by KROMACHEM LTD, or Chromazone® as a powder or a dispersion, or Thermobatch® or Thermostar®, from CHROMAZONE.

Piezochromic and Tribochromic Agents

A piezochromic agent can change color in the presence of a mechanical force.

An example of a piezochromic agent that may be mentioned is diphenylflavylene.

A tribochromic agent can change color in the presence of a mechanical force in a manner which is more durable than with piezochromic agents.

Reference may be made to International patent application WO-A-94/26729, the contents of which are hereby incorporated by reference.

Mechanoluminescent Agents

These agents are capable of emitting light when they receive a mechanical stress such as compression, shear, or friction.

The mechanoluminescent agent is preferably in the form of a particle which is insoluble in the cosmetic medium. The mean particle size is, for example, in the range 0.01 μm to 50 μm, preferably in the range 0.1 μm and 10 μm.

Mechanoluminescent materials that may be mentioned are as follows:

a) complexes and chelates of lanthanides such as those described in publications U.S. Pat. No. 6,071,632, US-A-2002/0015965 and WO-A-09/016,429, the contents of which are hereby incorporated by reference. The rare earths are preferably selected from europium, terbium, samarium, and dysprosium. In those materials, diketones are used as the ligand for the trivalent lanthanide salts. These materials are in an organic medium . . .

b) aluminates, silicates and aluminosilicates doped with rare earth ions such as those described in U.S. Pat. No. 6,280,655, EP-A-0 1 318 184, JP-A-2002/194349, JP-A-2004/59746, the contents of which are hereby incorporated by reference, in particular (Sr,Mg,Ba,Zn,Ca) Al₂O₄, (SrLa,SrY)Al₃O₇, (Sr₂,SrMg,SrCa,SrBa)Al₆O₁₁, Sr₂ (Mg,Al) (Al,Si)SiO₇, Sr(Zn,Mn,Fe,Mg)Si₂O₆. The elements shown in parentheses are partially or entirely interchangeable. Rare earth ions such as cerium, europium, samarium, neodymium, gadolinium, dysprosium, and terbium may be used, alone or as a mixture. Europium and dysprosium are preferred;

c) zinc sulfide, manganese sulfide, copper sulfide, cadmium sulfide or zinc oxide, optionally doped with transition metal ions or rare earth ions as described in the publications U.S. Pat. No. 6,117,574 and JP-A-2004/43656 the contents of which are incorporated by reference. Preferred transition metal ions are copper or manganese. Preferred rare earth ions are europium or cerium. Of these materials, ZnS:Mn is preferred.

The materials listed under b) and c) may be synthesized by a solid phase reaction involving dry mixing followed by heat treatment and high temperature sintering, or by a sol-gel process followed by drying, heating and sintering. As an example, the sintering temperature is more than 1000° C.

The materials listed under b) are preferred. Of these, SrAl₂O₄ and SrMgAl₁₀O₁₇ doped with rare metals are preferred.

The mechanoluminescent pigments SrAl₂O₄ doped with rare metal ions are sold with reference TAIKO-Ml-1 by TAIKO Refractories Co., Ltd. The particles of this pigment have a diameter in the range 5 μm to 10 μm and a green luminescence under a weak mechanical stress.

Solvatochromic Agents

A solvatochromic agent can change color in the presence of solvents. DC Red 27 is an example, this compound having an absence of color in an anhydrous formulation; adding water reveals a pink color.

Galenical Forms

The cosmetic composition may be in any galenical form which is normally used for topical application, in particular in the anhydrous form, in the solid or semi-solid form, in the form of an oily or aqueous solution, an oily or aqueous gel, an oil-in-water or water-in-oil emulsion, a multiple emulsion, a dispersion of oil in water by means of vesicles on the oil/water interface, or a spray, provided that the red highlights are conserved.

The composition may constitute any substance for application to the skin, e.g. a foundation, an eye contouring product, a blusher, an eye shadow, a makeup base, or a product for making up the body.

The composition of the invention may be obtained using conventional cosmetic preparation methods.

Packaging and Modes of Application

The composition may be packaged in any receptacle or on any surface provided for the purpose.

The composition of the invention may be in the solid form, especially as a cake or pearls or powder, a semi-solid, a liquid, a paste or a cream of greater or lesser fluidity.

The composition may be applied using an applicator, which may optionally be flocked, for example a foam, a tip, a brush, a felt tip, a spatula, a frit, a brush, a comb, or a woven or nonwoven fabric.

Application may also be made with the finger or by disposing the composition directly on the surface to be made up, for example by friction or spraying or by projection using a piezoelectric device, or by transferring a layer of composition disposed on an intermediate surface.

The composition may, for example, be applied in a thickness in the range 1 μm to 10 μm, for example.

As an example, the composition may be applied in a density in the range 1 mg/cm² [milligram per square centimeter] to 5 mg/cm².

If appropriate, the composition may be applied as a base coat covered with a layer of another composition (top coat) or as a top coat on a layer of another composition, or even between a base coat and a top coat in order, for example, to enhance the hold and/or gloss.

Magnetic Devices

The invention also provides a kit comprising a composition as defined above and at least one magnetic device for generating a magnetic field that makes it possible to displace and/or modify the orientation of the magnetic bodies.

The magnetic device may comprise a permanent magnet or an electromagnet powered by at least one optionally-rechargeable battery, for example. For a battery, the magnetic device may include a switch enabling the electromagnet to be powered selectively with electricity.

The magnetic device may be arranged so as to create a magnetic field of orientation that varies over time. When the magnetic device comprises a magnet, the device may, for example, include a motor enabling the magnet to be rotated. In a variant, the magnetic device may comprise a plurality of solenoids disposed so as to generate a rotating magnetic field when powered sequentially with electricity.

By way of example, a rotating magnetic field may make it possible to obtain a pattern presenting circular symmetry, e.g. a pattern giving the impression of a sphere in relief.

The electromagnet(s) may be powered continuously or intermittently, as desired by the user. In particular, the magnetic device may be arranged so that the electromagnets(s) need not be powered while the magnetic device is not correctly positioned close to the surface coated with the first composition.

The magnetic field is at least 50 milli teslas (mT), for example, and preferably at least 0.2 T, and preferably at least 1 T (10,000 Gauss).

In order to make it easier to apply the magnetic field, the magnetic device may include a member enabling it to be positioned relative to the surface on which the composition has been deposited. This makes it possible to prevent the magnetic device from accidentally coming into contact with the composition and/or makes it possible to center the pattern formed on the region under consideration.

In an implementation of the invention, the magnetic device is secured to an applicator that is used to apply the cosmetic composition. This makes it possible to reduce the number of objects that need to be manipulated by the user and makes it easier to apply makeup.

In another implementation of the invention, the magnetic device comprises a magnet mounted at a first end of a rod having a second end that is connected to a handle of an applicator that is used to apply the cosmetic composition.

The magnetic field may also be exerted by means of a magnetic structure, in particular a flexible structure, including alternate N and S poles. By way of example, such a structure may make it possible to form repeated patterns, e.g. stripes, on the composition.

Makeup Method

In another one of its aspects, the invention also provides a makeup method consisting in applying to the keratinous substances, using at least one cosmetic composition, at least one first interference pigment that, when the composition is applied to a surface, is capable of generating red highlights with an intensity that is greater than or equal to 3000 cd·m⁻² and with a dominant wavelength in the range 580 nm to 650 nm; and reflective particles that are capable of generating, on said surface, other highlights with an intensity that is greater than or equal to the intensity of the red interference pigment.

The first interference pigment and the reflective particles can be applied using the same composition.

The first interference pigment and the reflective particles can alternatively be applied using two different compositions that respectively contain the red interference pigment and the coloring agent that is sensitive to at least one external stimulus.

In another one of its aspects, the invention also provides a method of applying makeup to keratinous substances, the method comprising the following steps:

1)applying, to the keratinous substances, a layer of a composition as defined above,

2) subjecting the deposit to a magnetic field, thereby modifying the orientation and/or the position of at least a fraction of the magnetic bodies within the layer deposited in this way.

The present invention also provides a makeup method consisting in using at least one cosmetic composition to apply to the keratinous substances, an interference pigment that is red and that is capable of generating highlights with an intensity that is greater than or equal to 3000 cd·m⁻² and with a dominant wavelength in the range 580 nm to 650 nm, and magnetic bodies that present non-zero magnetic susceptibility.

The red interference pigment and the magnetic bodies can be applied using the same composition.

The red interference pigment and the magnetic bodies can alternatively be applied using two different compositions that respectively contain the red interference pigment and the magnetic bodies.

In another one of its aspects, the invention also provides a makeup method consisting in applying to the keratinous substances, using at least one cosmetic composition, at least one interference pigment that is red and that, once applied, is capable of generating highlights with an intensity of 3000 cd·m⁻² or more and with a dominant wavelength in the range 580 nm to 650 nm; and at least one reflective second pigment that is silvery or colored with a dominant wavelength λ₂ such that |λ₁−λ₂|≧50 nm, this second pigment having an average size that is 30 μm or more, better 40 μm.

The red interference pigment and the reflective second pigment can be applied using the same composition.

The red interference pigment and the reflective second pigment can alternatively be applied using two different compositions that respectively contain the red interference pigment and the reflective second pigment.

In another one of its aspects, the invention provides a makeup method consisting in applying to keratinous substances, by means of at least one cosmetic composition, at least one red interference pigment that, after application, can generate highlights with an intensity of 3000 cd·m⁻² or more and with a dominant wavelength in the range 580 nm to 650 nm and at least one coloring agent sensitive to at least one external stimulus.

The red interference pigment and the coloring agent which is sensitive to at least one external stimulus may be applied using the same composition.

The red interference pigment and the coloring agent that is sensitive to at least one external stimulus may also be applied via two different compositions respectively containing the red interference pigment and the coloring agent that is sensitive to at least one external stimulus.

Kit

In another one of its aspects, the invention also provides a makeup kit comprising:
a first composition comprising, in a cosmetically acceptable medium, at least one first interference pigment that, when the composition is applied to a surface, is capable of generating red highlights with an intensity that is greater than or equal to 3000 cd·m⁻² and with a dominant wavelength in the range 580 nm to 650 nm; and

a second composition comprising, in a cosmetically acceptable medium, reflective particles that are capable of generating, on said surface, other highlights with an intensity that is greater than or equal to the intensity of the red interference pigment.

In another one of its aspects, the invention also provides a makeup kit comprising:
a first composition comprising, in a cosmetically acceptable medium, an interference pigment that is red and that is capable of generating highlights with an intensity that is greater than or equal to 3000 cd·m⁻² and with a dominant wavelength in the range 580 nm to 650 nm; and

a second composition comprising, in a cosmetically acceptable medium, magnetic bodies that present non-zero magnetic susceptibility.

The second composition may be applied under or over the first.

In another one of its aspects, the invention also provides a makeup kit comprising:
a first composition comprising, in a cosmetically acceptable medium, at least one interference pigment that is red and that, when the composition is applied to a surface, is capable of generating red highlights with an intensity of 3000 cd·m⁻² or more and with a dominant wavelength in the range 580 nm to 650 nm; and
a second composition comprising, in a cosmetically acceptable medium, at least one reflective second pigment that is silvery or colored with a dominant wavelength λ₂ such that |λ₁−λ₂|≧50 nm, this second pigment having an average size that is 30 μm or more, better 40 μm.

In another one of its aspects, the invention provides a makeup kit comprising:
a first composition comprising, in a cosmetically acceptable medium, at least one red interference pigment that, when the composition is applied to a support, can generate highlights with an intensity of 3000 cd·m⁻² or more and with a dominant wavelength in the range 580 nm to 650 nm;
a second composition comprising, in a cosmetically acceptable medium, at least one coloring agent which is sensitive to at least one external stimulus.

### EXAMPLES

The amounts shown are by weight.

### Example 1

### Blusher

| | |
|---|---|
| Magnesium sulfate | 1.5 |
| Sodium carboxymethyl cellulose | 0.5 |
| Distearyl dimethyl ammonium modified | 1 |
| hectorite | |
| Cyclopenta dimethylsiloxane | 16 |
| Glycerol | 5 |
| Oxyethylene polymethylcetyl dimethyl | 9 |
| methylsiloxane/polyglycerol-4 | |
| isostearate/hexyl laurate mixture | |
| Water | 41.6 |
| Acetyl ethylene glycol stearate/glyceryl | 0.3 |
| tri-stearate mixture | |
| Poly dimethylsiloxane (viscosity: 5 cst) | 16 |
| Red interference pigments* | 5 |
| 1,5-pentanediol | 3 |

*Red interference pigment with silica core covered with a layer of iron oxide, sold by MERCK under reference XIRONA LE ROUGE.

The tone is perceived as being warmer and more radiant.

### Example 2

### Blusher

| | |
|---|---|
| Triethanolamine | 1 |
| Ethylene diamine tetraacetic acid, | 0.2 |
| disodium salt, 2H₂O | |
| Cross-linked carboxyvinyl homopolymer | 0.5 |
| Polyvinylpyrrolidone | 0.6 |
| Glycerol | 5.75 |
| Deionized water | 83.05 |
| 1,3 butylene glycol | 2 |
| Silica microsphere (3 μm) | 1.5 |
| Red interference pigment* | 5 |

*idem Example 1

The effect is substantially the same as for Example 1.

### Example 3

### Blusher

| | |
|---|---|
| Octyl-2 dodecanol | 10 |
| Ditertiobutyl 4-hydroxytoluene | 0.07 |
| Polybutene | 50 |
| (monoolefins/isoparaffins 95/5) | |
| (MW: 2060) | |
| Mixture of isopropyl, isobutyl, n- | 0.4 |
| butyl, p-hydroxybenzoates | |
| (40/30/30) | |
| Pentaerythrityl tetra-iso-stearate | 11.33 |
| Tridecyl tri-mellitate | 13 |
| 2-decyl tetradecanoic acid | 13 |
| triglyceride (GUERBET C24) | |
| Magnetic pigment* | 0.2 |
| Red interference pigment** | 2 |

*Acicular soft iron pigment
**Interference pigment comprising a silica core covered with a layer of iron oxide, sold by MERCK under reference XIRONA LE ROUGE.

### Example 4

Base layer

| | |
|---|---|
| Tri-decyl tri-mellitate | 11 |
| Liquid lanolin | 10 |
| Iso-stearyl malate | 13 |
| Acetylated lanolin | 10 |
| Lauric/palmitic/cetyl/stearic acid triglycerides | 5 |
| 50/20/10/10 | |
| Microcrystalline wax (C20-C60) | 3 |
| Protected isopropyl lanolate | 10 |
| 2-Octyldecanol | 16 |
| Phenyl trimethylsiloxy trisiloxane (VISCOSITY: | 4 |
| 20 CST - MW: 372) | |
| Polyethylene wax (MW: 500) | 8 |
| Pigment DC RED7 CI 15850 | 5 |

Covering layer

| | |
|---|---|
| Octyl-2 dodecanol | 10 |
| Ditertiobutyl 4-hydroxytoluene | 0.07 |
| Polybutene | 50 |
| (monoolefins/isoparaffins 95/5) | |
| (MW: 2060) | |
| Mixture of isopropyl, isobutyl, n- | 0.4 |
| butyl p-hydroxybenzoates | |
| (40/30/30) | |
| Pentaerythrityl tetra-iso-stearate | 11.33 |
| Tridecyl tri-mellitate | 12 |
| 2-decyl tetradecanoic acid | 11 |
| triglyceride (GUERBET C24) | |
| Colored interference pigment* | 5 |

*Red interference pigment comprising a silica core covered with a layer of iron oxide Fe₂O₃, available from MERCK under reference XIRONA LE ROUGE.

The base layer forms a continuous uniform background. By means of the covering layer, a shimmering effect is obtained that is all the more visible since the coloring agent of the base layer is separate from the red interference pigment.

Clearly, the invention is not limited to the examples given above. The expression “comprising a” is synonymous with “comprising at least a” and “in the range” means that the limits are included.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method of making-up dark skin, the method comprising the step consisting in applying to the skin a cosmetic composition comprising, in a cosmetically acceptable medium, at least one red interference pigment which, when the composition is applied to the skin, is capable of generating highlights with an intensity of 3000 cd·m⁻² or more and with a dominant wavelength in the range 580 nm to 650 nm.

2. A method according to claim 1, the interference pigment being capable of generating highlights with an intensity of 3400 cd·m⁻² or more.

3. A method according to claim 1, the composition presenting saturation C* that is 30 or more.

4. A method according to claim 3, the composition being 35 or more.

5. A method according to claim 1, the composition presenting a hue angle h° in the range 10° to 30°.

6. A method according to claim 5, the hue angle being in the range 15° to 25°.

7. A method according to claim 1, the amount of red interference pigment lying in the range 0.1% to 15% by weight when the composition is liquid, and lying in the range 0.1% to 60% by weight when the composition is solid.

8. A method according to claim 1, the composition having no coloring agent other than the red interferential pigment.

9. A method according to claim 1, the size of the red interference pigment being 30 μm or more.

10. A method according to claim 1, the size of the red interference pigment being 40 μm.

11. A method according to claim 1, the size of the red interference pigment being in the range 30 μm to 80 μm.

12. A method according to claim 1, the red interference pigment including an inorganic core.

13. A method according to claim 12, the core being made of silica, glass, or mica.

14. A method according to claim 1, including a surface layer of a metal oxide.

15. A method according to claim 14, the metal oxide comprising iron oxide Fe₂O₃.

16. A method according to claim 1, the composition comprising a colorant.

17. A makeup composition comprising, in a cosmetically acceptable medium, at least one colorant, and at least one red interference pigment which, when the composition is applied to the skin, is capable of generating highlights with an intensity of 3000 cd·m⁻² or more and with a dominant wavelength in the range 580 nm to 650 nm.

18. A composition according to claim 17, the colorant having a dominant wavelength in the range 580 nm to 650 nm.

19. A composition according to claim 17, the colorant being hydrosoluble.

20. A composition according to claim 17, the colorant being liposoluble.

21. A composition according to claim 17, the colorant being selected from amongst caramel, beetroot juice and carmine, betanin (beetroot), copper chlorophyllin, methylene blue, anthocyanins (enocianin, black carrot, hibiscus, elder), riboflavin, Sudan red, β-carotene, carotenoids, lycopene, palm oil, Sudan brown, quinoline yellow and xanthophylles (capsanthin, capsorubin, lutein), curcumin, anthocyans from flowers or fruit and their derivatives, flavonoids and tannins extracted from native or fermented vegetables, juglone, lawsone, fermented soya extracts, algae, fungi, micro-organisms, Flavylium salts not substituted in the 3-position, extracts from Gesneria Fulgens, Blechum Procerum, Saxifraga and pigments which can be obtained by extraction with an organic or hydro-organic solvent from a culture medium of micromycetes of the monascus Monascus type, synthetic liposoluble colorants such as DC Red 17, DC Red 21, DC Red 27, DC Green 6, DC Yellow 11, DC Violet 2, and DC Orange 5, and synthetic hydrosoluble colorants such as FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, and FDC Blue 1.

22. A composition according to claim 17, the size of the red interference pigment being in the range 30 μm to 80 um.

23. A composition according to claim 17, the red interference pigment being capable of creating highlights with an intensity of 3500 cd·m⁻² or more, the composition not containing, in the medium, white fillers or solid bodies that generate a color by absorption, or, when the composition does contains them, the total amount of such solid bodies being 1% or less by weight relative to the total weight of the composition.

24. A composition according to claim 17, comprising reflective particles that are capable of generating, on said surface, other highlights with an intensity that is greater than or equal to the intensity of the red interference pigment.

25. A composition according to claim 17, presenting a turbidity index of 100 NTU or less.

26. A composition according to claim 17, comprising magnetic bodies presenting non-zero magnetic susceptibility.

27. A composition according to claim 17, comprising a reflective second pigment that is silvery or that is colored with a dominant wavelength λ₂ such that |λ₁−λ₂|≧50 nm, this second pigment having an average size that is 30 μm or more.

28. A composition according to claim 27, the second pigment having an average size that is 40 μm.

29. A composition according to claim 17, comprising at least one coloring agent that is sensitive to at least one external stimulus.

30. A set of at least two cosmetic compositions according to claim 17, the saturation difference between two compositions of the set being 2 or less, the red interference pigment in said two compositions being at concentrations that differ by at least 1%.
